(19) United States
(12) Patent Application Publication
(10) Pub. No.: US 2024/0002939 A1
(43) Pub. Date: Jan. 4, 2024

(54) METHODS OF DIAGNOSTIC OF LIVER FIBROSIS

(71) Applicant: GENFIT, LOOS (FR)

(72) Inventors: SUNEIL HOSMANE, ARLINGTON, MA (US); JÉRÉMY MAGNANENSI, LILLE (FR); YACINE HAJJI, LILLE (FR)

(21) Appl. No.: 18/250,990

(22) PCT Filed: Oct. 29, 2021
(86) PCT No.: PCT/EP2021/080096

§ 371 Date: Apr. 28, 2023

(30) Foreign Application Priority Data
Oct. 30, 2020 (EP) 20306303.7

(51) Int. Cl. C12Q 1/6883; C12Q 1/6851; G01N 33/68

(52) U.S. Cl. CPC C12Q 1/6883; C12Q 1/6851; G01N 2800/085; C12Q 2600/178; G01N 33/6893

(57) ABSTRACT

The invention relates to a method for the identification of NAFLD patients likely to develop advanced liver fibrosis within 52 weeks.

### TECHNICAL FIELD

The invention relates to a method for the identification of NAFLD (Non-Alcoholic Fatty Liver Disease) patients likely to develop advanced liver fibrosis within 52 weeks.

### BACKGROUND

Abnormal and exaggerated deposition of extracellular matrix is the hallmark of all fibrotic diseases, including liver, pulmonary, kidney or cardiac fibrosis. The spectrum of affected organs, the progressive nature of the fibrotic process, the large number of affected persons, and the absence of symptoms before the disease becomes life-threatening pose an enormous challenge.

When the liver is damaged, fibrous layers are formed which become scar tissue in the liver. This early phase of damage is called fibrosis.

Several types of liver diseases exist that can cause fibrosis. These include:
autoimmune hepatitis
biliary obstruction
nonalcoholic fatty liver disease, which includes nonalcoholic fatty liver (NAFL) and nonalcoholic steatohepatitis (NASH)
viral hepatitis B and C
alcoholic liver disease
Hepatitis D: This type of hepatitis can also cause cirrhosis. It is often seen in people who already have hepatitis B.
Damage to the bile ducts, which function to drain bile: One example of such a condition is primary biliary cirrhosis.
Disorders that affect the body's ability to handle iron and copper: Two examples are hemochromatosis and Wilson's disease.
Medications (acetaminophen, some antibiotics, and some antidepressants, can lead to cirrhosis).

The most common cause of liver fibrosis is nonalcoholic fatty liver disease (NAFLD), while the second is alcoholic liver disease due to long-term excesses of drinking alcohol.

Liver Damage can range from:
little to mild damage
mild to moderate damage (fibrosis)
moderate to severe damage (fibrosis to compensated cirrhosis)
severe to liver failure (decompensated cirrhosis)

“Liver fibrosis” refers to the presence of fibrous connective tissue at microscopic examination of a stained (H&E, trichrome or picrosirius red staining) slice of a liver biopsy. In the context of the present invention, the term “fibrosis stage” denotes the localization and extent of liver fibrosis at histological exam, as follows:
Perisinusoidal or periportal fibrosis 1
Mild perisinusoidal fibrosis (zone 3) 1a
Moderate perisinusoidal fibrosis (zone 3) 1b
Portal/periportal fibrosis 1c
Perisinusoidal and portal/periportal fibrosis 2
Bridging fibrosis 3
Cirrhosis 4

(Kleiner et al., Design and Validation of a Histological Scoring System for Nonalcoholic Fatty Liver Disease Hepatology, 2005; 41:1313-1321).

In many cases, there are little to no symptoms with fibrosis until liver damage becomes severe enough (cirrhosis), in which the patient can experience severe fatigue, appetite loss, difficulty thinking clearly, confusion, swelling in the abdomen (ascites) and legs, skin itching, and bleeding in the digestive tract, etc.

Mild liver damage from fibrosis can be reversed if the cause is found and eliminated before too much damage occurs, but if scarring continues over a long period of time, fibrosis becomes permanent. Damage continues to form bands throughout the liver, destroying the liver's internal structure and destroys the liver's ability to regenerate and impairs liver function, leading to cirrhosis.

Complications from liver damage (scar tissue) can interrupt liver function. Scar tissue replaces healthy liver cells which are needed to perform liver functions. Scar tissue interrupts with blood flow to the liver. Without enough blood in the liver, the cells die and more scar tissue is formed.

The more scar tissue is formed, the most severe complications it can cause.

Ultimately, if a person's fibrosis progresses to cirrhosis and liver failure, he or she can have complications such as:

ascites (severe buildup of fluid in the abdomen)

hepatic encephalopathy (buildup of waste products that causes confusion)

hepatorenal syndrome

portal hypertension

variceal bleeding.

The most significant complication of liver fibrosis can be liver cirrhosis, or severe scarring that makes the liver so damaged that a person will become sick. Usually, this takes a long time to occur, such as over the course of one or two decades.

Liver cirrhosis is one of the leading causes of death worldwide. Cirrhosis can lead to liver tumors that are cancerous, liver failure and the need for liver transplant. Therefore, it is important that a person be diagnosed and treated for liver fibrosis as early as possible before it progresses to liver cirrhosis. An estimated 6 to 7 percent of the world's population has liver fibrosis and does not know it because these persons don't have symptoms.

Because this disease can be potentially reversed if diagnosed early enough, or at least its consequences limited, it seems to be crucial to be able to provide the medical field with adapted tools allowing such an early, rapid and precise diagnostic.

Although several attempts were made to propose non-invasive methods for diagnosing and determining the severity of liver fibrosis, as of today histological analysis of liver biopsies remains the optimal approach for assessing the stage of fibrosis. However, liver biopsy has a number of obvious drawbacks. First, the material collected in liver biopsy represents only a very small part of the liver of the diagnosed subject, thereby raising doubts on whether the collected sample is representative of the global state of the subject's organ. Moreover, liver biopsy is a very invasive procedure that may be cumbersome, worrisome and painful for the patient, and which raises concerns about morbidity and mortality. At last, in view of the foregoing, liver biopsy cannot reasonably be proposed as a routine procedure for determining whether a person has a fibrosis.

These drawbacks of biopsy-based diagnosis led to an active development of non-invasive methods for the detection of NASH (nonalcoholic steatohepatitis). For example, WO2017046181 and WO2017167934 provide non-invasive diagnosis based on the measure of the level of circulating biomarkers.

Another option is an imaging test known as transient elastography. This is a test that measures how stiff the liver is. When a person has liver fibrosis, the scarred cells make the liver stiffer. This test uses low-frequency sound waves to measure how stiff liver tissue is. However, it is possible to have false positives where the liver tissue may appear stiff.

### SUMMARY OF THE INVENTION

The present invention relates to a method for the identification of a subpopulation of NAFLD subjects who are at risk of progressing to advanced fibrosis (F≥3) within the course of 52 weeks. Subjects identified as such are qualified as “fast progressors”.

It is shown in the experimental part below that the method of the invention has a better prognostic value than other tests available in the art, such as FIB-4 (Fibrosis 4), ELF (Enhanced Liver Fibrosis) and NFS (NAFLD fibrosis score), for identifying fast progressors.

In a particular embodiment, the method of the invention further comprises a step of measuring liver fibrosis of said subject with a physical method. In particular, the method can comprise a step of measuring liver stiffness of said subject. In a further particular embodiment, liver stiffness is determined by measuring the difference in velocity of elastic shear wave propagation in the liver.

### DETAILED DESCRIPTION OF THE INVENTION

According to the present invention, the terms “fibrosis”, “fibrotic disease”, “fibrotic disorder” and declinations thereof denote a pathological condition of excessive deposition of fibrous connective tissue in the liver. More specifically, fibrosis is a pathological process, which includes a persistent fibrotic scar formation and overproduction of extracellular matrix by the connective tissue, as a response to tissue damage. Physiologically, the deposit of connective tissue can obliterate the architecture and function of the underlying organ or tissue.

According to the invention, the term “non-alcoholic steatohepatitis”, or NASH, refers to a NAFLD condition characterized by the concomitant presence of liver steatosis, hepatocyte ballooning and liver inflammation at histological examination, in the absence of excessive alcohol consumption and after excluding other liver diseases like viral hepatitis (HCV, HBV). According to the invention, the term “steatosis” refers to the process describing the abnormal retention of lipids or fat accumulation within the liver. According to the present invention, the term “hepatocellular ballooning” is usually defined, at the light microscopic level, based on hemotoxylin and eosin (H&E) staining, as cellular enlargement 1.5-2 times the normal hepatocyte diameter, with rarefied cytoplasm. It refers more generally to the process of hepatocyte cell death. According to the present invention, the term “lobular inflammation” refers to the presence of lobular inflammatory foci (grouped inflammatory cells) at microscopic examination of a hematoxylin and eosin (H&E) stained slice of a liver biopsy.

According to the present invention, the “NAFLD-Activity score” or “NAS” refers to the sum of steatosis, hepatocellular ballooning, lobular inflammation scores, as follows:

S: Steatosis score: 0: <5%; 1: 5-33%; 2: 34-66% and 3: >66%;

LI: Lobular Inflammation score (foci/x20 field): 0: none; 1: <2; 2: 2-4 and 3: >4;

HB: Ballooning degeneration score: 0: none; 1: few; 2: many cells/prominent ballooning.

Therefore, NASH refers to a NAFLD condition characterized by the following liver biopsy-derived grades: NAS3, with at least 1 point in steatosis, at least 1 point in lobular inflammation and at least 1 point in the hepatocyte ballooning scores.

More severe forms of NASH are also characterized by higher grades in one of the S, LI and HB scores described above, and/or the presence of liver fibrosis.

As mentioned above, the fibrosis stages range from F=0 (or F0), i.e. no fibrosis, to F4, i.e. cirrhosis. In the context of the present invention, a fibrosis stage of F≥3 (i.e. F3 or F4) is referred to herein as “advanced fibrosis”.

The subject identified as a fast progressor according to the method of the present invention may be a subject having a fibrosis stage of 0, 1 or 2 and a condition selected in the group consisting of NAFLD, non-alcoholic steatohepatitis (NASH), proliferative fibrosis, biliary obstruction, alcohol or drug-induced liver fibrosis, liver cirrhosis, infection-induced liver fibrosis, in particular viral infection like Hepatitis A, B or C, radiation or chemotherapeutic-induced fibrosis, chronic fibrosing cholangiopathies such as Primary Sclerosing Cholangitis (PSC), Primary Biliary Cholangitis (PBC), biliary atresia, hemochromatosis, Wilson's disease and medication-induced liver fibrosis. In a particular embodiment, the subject identified as a fast progressor according to the method of the present invention may be a subject with NASH and fibrosis stage of 0, 1 or 2. The present invention more particularly provides a method to determine the likelihood of said subject to progress towards advanced fibrosis within 52 weeks from the determination.

In the method of the present invention, the level of at least 4 circulating markers is measured from a blood-derived sample from the subject. Said at least 4 circulating markers are: hsa-miR34a, alpha 2 macroglobulin (A2M), YKL40 and glycated hemoglobin (HbA1c).

The measure of the level of these markers is conducted in a blood-derived sample of the subject, such as blood, serum or plasma, in particular platelet-free plasma, e.g. a cell-free, citrate-derived platelet-free plasma sample. In a particular embodiment, the level of hsa-miR34a, A2M and YKL40 is measured from one or more serum sample(s) from the subject. In another particular embodiment, the level of HbA1c is measured from a blood sample of the subject.

In a particular embodiment, the level of hsa-miR34a, A2M, YKL40 and HbA1c is measured as described in WO2017167934.

It should be understood that in all embodiments and variants disclosed herein, hsa-miR34a can more particularly be hsa-miR34a-5p. In the present application, the combination of hsa-miR34a-5p, alpha 2 macroglobulin (A2M), YKL40 and glycated hemoglobin (HbA1c) is also referred to as NIS4™.

In a particular embodiment, the levels of the circulating markers as measured herein are used in a logistic function to calculate a score, for example as provided in application WO2017167934. Briefly, the logistic function can be determined thanks to data obtained from reference subjects with NASH, who progressed to advanced fibrosis from a F0, F1 or F2 stage at inclusion. Such data may have been obtained:

by a first set of measurements of circulating levels of the markers when the reference subjects were at the F0, F1 or F2 stage, as determined by a liver biopsy, and

measurements carried out 52 weeks after said first set of measurements, in the same reference subjects, of circulating levels when said reference subjects were at F≥3 stage as determined by another liver biopsy.

One skilled in the art can use the information provided in the experimental part herein and the teaching of WO2017167934 to determine the logistic function and cutoffs relevant to the required test sensitivity, specificity, positive predictive value and/or negative predictive value.

According to a particular embodiment, the score is defined as a logistic function derived from a bootstrap model:

$$S ∼ \frac{{e}^{Y}}{1 + {e}^{Y}}$$

wherein:

Y=k+a*A+b*B+c*C+d*D

wherein:

S is the score;

A is the serum level of hsa-miR-34a (in particular hsa-miR-34a-5p) in Cq;

B is the serum level of alpha 2 macroglobulin in g/L;

C is the serum level of YKL-40 in pg/ml,

D is the level of HbA1c in percent (e.g. D is equal to 10 if measured HbA1c percentage is 10%);

k is the constant of the logistic function

a is a coefficient associated to the serum level of hsa-miR-34a (in particular hsa-miR-34a-5p);

b is a coefficient associated to the serum level of alpha 2 macroglobulin;

c is a coefficient associated to the serum level of YKL-40;

d is a coefficient associated to the level of HbA1c.

In a further particular embodiment, derived from the bootstrap model as described in the experimental part of application WO2017167934:

k is a number comprised between 9.51 and 34.37;

a is a number comprised between −1.17 and −0.47;

b is a number comprised between 0.02 and 0.84;

c is a number comprised between 6.10E-06 and 2.09E-05; and

d is a number comprised between 0.07 and 0.89.

As mentioned above, one skilled in the art can use the information provided herein and the teaching of WO2017167934 to determine the logistic function and cutoffs relevant to the required test sensitivity, specificity, positive predictive value and/or negative predictive value. In a particular embodiment, the low cut-off is 0.36 and the high cut off is 0.63. A shown in the experimental part, thanks to this particular embodiment, subjects with a score lower than 0.36 can be identified as having a low likelihood of progressing to advanced fibrosis (ruled out) with a probability of progression of 2% only, whereas those with a score higher or equal to 0.63 can be identified as having a high likelihood of progressing to advanced fibrosis (ruled in) with a probability of progression to advanced fibrosis in 52 weeks of 38% (positive predictive value of 38% (23-56)), 88% (81-92) specificity and 69% (44-86) sensitivity.

In a particular embodiment, the method further comprises the measure of liver fibrosis of said subject with a physical method. This measure can be conducted thanks to a number of methods well known in the art. Illustrative methods include, without limitation, medical imaging and/or clinical measurement. In a particular embodiment, the physical method is elastometry. Elastometry method can further particularly be selected from the group consisting of Acoustic Radiation Force, Impulse imaging (ARFI imaging), transient elastography (TE) and MRI stiffness. In a particular embodiment of the invention, the physical method is transient elastography, which measures the difference in velocity of elastic shear wave propagation in the liver. According to a preferred method, transient elastography (such as FIBROSCAN®) is used, a technique used to assess liver hardness or stiffness, measured in kilopascal (kPa) and correlated to fibrosis, without invasive investigation. TE results (such as FIBROSCAN® results) can range from 2.5 kPa to 75 kPa. Between 90-95% of healthy subjects without liver disease will have a liver stiffness measurement <7.0 kPa. In a particular embodiment, the method can comprise a step of measuring liver stiffness of said subject. In a further particular embodiment, liver stiffness is measured by measuring the difference in velocity of elastic shear wave propagation in the liver.

The invention is further described with reference to the following, non-limiting, examples.

### EXAMPLES

### Example 1: Histological Progression to Advanced Fibrosis and Cirrhosis in NASH

The analysis was performed on a subpopulation from phase 2 clinical trial GOLDEN-505 (NCT01694849) study including 161 patients with histologically confirmed NASH, NAFLD activity score (NAS)≥3, and fibrosis stage 0-2 who had both baseline and end-of-study (week 52) biopsies. GOLDEN-505 was a multicentre, randomized, double blind, placebo-controlled study to evaluate the efficacy and safety of Elafibranor (1-[4-methylthiophenyl]-3-[3,5-dimethyl-4-carboxydimethylmethyloxy phenyl]prop-2-en-1-one) once daily in patients with Non-Alcoholic Steatohepatitis (NASH).

To monitor histological progression of the disease, an inclusion liver biopsy and a biopsy at the end of the 1-year treatment were used for examination and scoring of histological lesions. Histologic scoring according to NASH-CRN system (NASH Clinical Research Network) was centralized and performed by a pathologist.

Patients characteristics were compared between “fast progressors” patients who transitioned from NASH (NAS≥3) with F0 to F2 fibrosis to advanced (F≥3) fibrosis in 52 weeks (n=16) and patients who did not progress to F≥3 (n=145).

Hsa-miR-34a-5p, alpha-2-macroglobulin, YKL-40, and hemoglobin A1c were quantified before algorithm calculation in order to determine NIS4™ score.

Briefly, patient's blood was collected in serum separating tube (SST) for the measure of YKL-40, hsa-miR-34a-5p levels, and alpha2-macroglobulin A2M. Blood was also collected in EDTA collection tube for HbA1c measure. YKL40 (also referred to as CHI3L1) was quantitatively determined by an ELISA (Human Chitinase 3-like 1 Immunoassay Quantikine® ELISA Catalog Number DC3L10). Values were expressed as ng/mL. Alpha 2 macroglobulin levels were measured by nephelometry on a BN II system (Siemens Healthcare). Values were expressed as g/L. HbA1c was measured by ion-exchange high performance liquid chromatography (HPLC) method (Menarini HA-8160 HbA1c auto-analyzer) and reported as a percentage of total haemoglobin.

Total RNA containing preserved miRNAs was extracted from 100 μl individual serum using miR-VanaParis extraction kit (AM1556, Ambion, Life Technologies, Carlsbad, CA) according to the manufacturer's instructions. In order to monitor extraction efficiency and for the minimization of sample-to-sample variation, i) a synthetic C. elegans miR-39 [3,125 fmoles] (MSY0000010, Qiagen, Venlo, The Netherlands) was added to each sample prior to RNA extraction and ii) a standard serum with a known miR-34a Cq value was processed at the same time of tested samples. The washing steps were then performed using miR-VanaParis washing solutions (8680G & 8543G14 Ambion) and centrifugation to avoid ethanol carryover. The total RNA including miRNAs was eluted in DNAse/RNAse-free water via centrifugation and immediately stored at −80° C. until use. A fixed volume of 5 μl of total RNA from serum samples or synthetic hsa-miRNA-34a (single strand sequence=5′Phos-UGGCAGUGUCUUAGCUGGUUGU-3′ (SEQ ID NO:1); Integrated DNA Technologies) diluted to 3.125 fmol/mL (used for standard curve construction and miR-34a copies number calculation) were concomitantly reverse transcribed using TaqMan MicroRNA Reverse transcription Kit (4366597, Applied Biosystems). Reverse transcription reaction was carried out in a final mixture of 15 μL containing 10 μL of TaqMan MicroRNA Assay 5X and incubated in a thermocycler GeneAmp® PCR System 9400 from Applied Biosystem. cDNAs were stored in low binding tubes at −20° C. until further use.

Expression of mature miRNAs was quantified according to the manufacturer's instructions using the Taqman miRNA RT-qPCR Assay 20X and TaqMan Universal Master Mix II, no Uracil-N-Glycosidase (UNG) 2X (Applied Biosystems). A fixed volume of 5 μL total RNA was used as a template for the qPCR assay using a CFX96TM Real-Time System. The hsa-miR-34a-5p TaqMan assay was used. The RT product from synthetic miRNAs was serially diluted and PCR was performed on all samples (standards and serum-derived RNA). Standard curve was performed in duplicate and used to convert Cq data in copies/μL. The Cq Determination mode was Regression. Transcript abundance is expressed in Cq.

The sequences of mature miRNA and Taq Man assay ID are reported in the following table:

| miRNA ID | Sequence | miRbase Number | Assay ID |
|---|---|---|---|
| cel-miR-39-3p | UCACCGGGUGUAAAUCAGCUUG | MIMAT0000010 | 000200 |
| | (SEQ ID NO: 2) | | |
| hsa-miR-34a-5p | UGGCAGUGUCUUAGCUGGUUGU | MIMAT0000255 | 000426 |
| | (SEQ ID NO: 1) | | |

Data used in the construction of the algorithm were in Cq format.

NIS4™ score was then calculated as provided in application WO2017167934, defined as a logistic function with the serum level of has-miR-34a-5p expressed in Cq unit.

In order to compare NIS4™ with other non-invasive scores, the following non-invasive scores stratified by established clinical cut-offs were assessed and used as prediction models:
Fibrosis-4 [FIB-4; Age, AST, ALT, Platelets]
NAFLD fibrosis score [NFS; Age, BMI, IGF/Diabetes Status, AST, ALT, Platelets, Albumin]
Enhanced liver fibrosis [ELF™; hyaluronic acid (HA), procollagen III amino-terminal peptide (PIIINP), and tissue inhibitor of matrix metalloproteinase (TIMP-1)].

Data and scores from patients group who did progress to F≥3 were compared with data from the patients group who did nor progress to F≥3 by using Chi2 or Wilcoxon p values.

TABLE 1
Patients characteristics.

| | | Patients Who | Patients Who | Chi2 or Wilcoxon |
|---|---|---|---|---|
| | | Did Progress | Did Not Progress | p values for both |
| | All Patients | to F ≥ 3 | to F ≥ 3 | group comparison |
| n | 161 | 16 | 145 | |
| Sex, male, % (n) | 53% (85) | 62% (10) | 52% (75) | 0.5785 |
| Age (years), mean ± SD | 51.48 ± 11.59 | 61.31 ± 9.41 | 50.4 ± 11.32 | 0.0002 |
| BMI (kg/m²), mean ± SD | 31.06 ± 4.83 | 33.01 ± 4.64 | 30.84 ± 4.82 | 0.0565 |
| Obese*, % (n) | 51% (82) | 69% (11) | 49% (71) | 0.2154 |
| Prediabetes†, % (n) | 15% (24) | 12% (2) | 15% (22) | 1 |
| Type 2 diabetes, % (n) | 32% (51) | 50% (8) | 30% (43) | 0.1685 |
| Dyslipidaemia‡, % (n) | 49% (78) | 50% (8) | 49% (70) | 1 |
| Arterial hypertension, % (n) | 49% (78) | 81% (13) | 45% (65) | 0.0142 |
| No metabolic risk factor§, % (n) | 13% (21) | 6% (1) | 14% (20) | 0.6396 |
| ALT (IU/L), mean ± SD | 61.19 ± 38.42 | 72.5 ± 31.19 | 59.94 ± 39.03 | 0.0258 |
| AST (IU/L), mean ± SD | 38.81 ± 23.3 | 57.94 ± 41.19 | 36.7 ± 19.52 | 0.0007 |
| Glucose (mmol/L), mean ± SD | 5.84 ± 1.66 | 6.36 ± 1.73 | 5.78 ± 1.65 | 0.1464 |
| HbA1c (%), mean ± SD | 5.94 ± 0.8 | 6.49 ± 0.94 | 5.88 ± 0.76 | 0.0038 |
| TG (mmol/L), mean ± SD | 1.84 ± 1.05 | 2.04 ± 1.19 | 1.82 ± 1.04 | 0.4977 |
| HDL-C (mmol/L), mean ± SD | 1.26 ± 0.33 | 1.34 ± 0.4 | 1.25 ± 0.32 | 0.3919 |
| LDL-C (mmol/L), mean ± SD | 2.89 ± 0.94 | 2.78 ± 0.77 | 2.91 ± 0.95 | 0.6694 |
| Fibrosis Stage, mean ± SD | 1.14 ± 0.71 | 1.88 ± 0.34 | 1.06 ± 0.7 | <0.0001 |
| NAS, mean ± SD | 4.88 ± 1.22 | 5.56 ± 1.21 | 4.81 ± 1.2 | 0.0187 |
| Non-invasive Diagnostics | | | | |
| FIB-4, mean ± SD | 1.22 ± 0.64 | 2.05 ± 1.03 | 1.13 ± 0.51 | 0.0001 |
| NFS, mean ± SD | −1.81 ± 1.33 | −0.74 ± 1.14 | −1.93 ± 1.3 | 0.0005 |
| ELF ™, mean ± SD | 9.13 ± 0.80 | 9.87 ± 0.67 | 9.04 ± 0.77 | 0.0001 |
| NIS4 ™, mean ± SD | 0.38 ± 0.23 | 0.67 ± 0.25 | 0.35 ± 0.21 | <0.0001 |
| miR-34a-5p (cq), mean ± SD | 31.87 ± 0.89 | 30.83 ± 1.47 | 31.98 ± 0.73 | <0.0001 |
| YKL-40 (ng/ml), mean ± SD | 59.29 ± 48.02 | 99.56 ± 79.96 | 54.85 ± 41.38 | 0.0077 |
| A2M (g/L), mean ± SD | 2.12 ± 0.74 | 2.82 ± 0.99 | 2.05 ± 0.67 | 0.0032 |

A2M: alpha-2-Macroglobulin.
ALT = alanine aminotransferase.
AST = aspartate aminotransferase.
BMI = body-mass index.
HbA1c = glycated haemoglobin.
NAS = non-alcoholic fatty liver disease activity score.
NASH = non-alcoholic steatohepatitis.
*BMI ≥30 kg/m².
†Fasting plasma glucose concentration between 5.6 mmol/L and 7.0 mmol/L, and not classified as type 2 diabetes.
‡Determined by use of dyslipidaemia medication.
§None of the following metabolic risk factors: obesity, prediabetes, type 2 diabetes, dyslipidaemia, or arterial hypertension.

“Fast Progressor” patients tended to have worse clinical and biochemical features as compared to non-fast progressing patients (Table 1), including:
Older age
Higher metabolic co-morbidities including obesity, type 2 diabetes mellitus (T2DM), and hypertension
Higher ALT, AST
Higher mean non-invasive scores (FIB-4, NFS, NIS4™, and ELF™)
   Patients who progressed to advanced fibrosis score were then categorized by score zone.

The High score zone was determined for scores above the high cut off for rule-in, the Indeterminate zone was defined when the score is between the high and the low cut off, and the Low score zone was determined when the score is lower than the Low cut off for rule-out. To enable real-world clinical use, a low cutoff was established at less than 0.36 to provide a rule-out decision with 81.5% sensitivity, 63% specificity, and 77.9% NPV. In addition, a high cutoff was established at 0.63 or higher to enable a rule-in decision with 87.1% specificity, 50.7% sensitivity, and 79.2% PPV (Harrison et al. (The Lancet Gastroenterology & Hepatology, volume 5, issue 11, p.970-985, Nov. 1, 2020; published online Aug. 3, 2020)

The NIS4™ score was calculated according to the method provided in W02017167934 and Harrison et al. (The Lancet Gastroenterology & Hepatology, volume 5, issue 11, p.970-985, Nov. 1, 2020; published online Aug. 3, 2020). Briefly, a logistic function derived from a bootstrap model was defined with the following features, adapted to the sought specificity, sensitivity, PPV and NPV of the low and high cutoffs defined above:

$$S ∼ \frac{{e}^{Y}}{1 + {e}^{Y}}$$

wherein:

Y=k+a*A+b*B+c*C+d*D
wherein:
S is the NIS4™ score;
A is the serum level of hsa-miR-34a (in particular hsa-miR-34a-5p) in Cq;
B is the serum level of alpha 2 macroglobulin in g/L;
C is the serum level of YKL−40 in pg/ml,
D is the level of HbA1c in percent (e.g. D is equal to 10 if measured HbA1c percentage is 10%);
k is a number comprised between 9.51 and 34.37;
a is a number comprised between −1.17 and −0.47;
b is a number comprised between 0.02 and 0.84;
c is a number comprised between 6.10E-06 and 2.09E-05; and
d is a number comprised between 0.07 and 0.89.

Absolute number and relative percentage of the «fast progressor» patients who transitioned from NASH (NAS≥3) and F0 to F2 fibrosis to advanced (F≥3) fibrosis were quantified for each test/score zone for NIS4™, FIB-4, NFS, and ELF as shown in Table 2.

TABLE 2
Categorization of patients who progressed to advanced fibrosis by score zone.

| | | | Number of Fast | % of Fast |
|---|---|---|---|---|
| Score or | | | Progressors | Progressors |
| Technology | Score Zone | Score Range | (to F ≥ 3) | (to F ≥ 3) |
| NIS4 ™ | High | ≥0.63 | 11 | 69% (11/16) |
| NIS4 ™ | Indeterminate | 0.36 ≤ NIS4 < 0.63 | 3 | 19% (3/16) |
| NIS4 ™ | Low | <0.36 | 2 | 13% (2/16) |
| FIB-4 | High | ≥2.67 | 4 | 25% (4/16) |
| FIB-4 | Indeterminate | 1.3 ≤ FIB-4 < 2.67 | 9 | 56% (9/16) |
| FIB-4 | Low | <1.3 | 3 | 19% (3/16) |
| NFS | High | ≥0.676 | 2 | 13% (2/16) |
| NFS | Indeterminate | −1.455 ≤ NFS < 0.676 | 9 | 56% (9/16) |
| NFS | Low | <−1.455 | 5 | 31% (5/16) |
| ELF ™ | High | ≥9.8 | 9 | 56% (9/16) |
| ELF ™ | Indeterminate | 7.7 ≤ ELF < 9.8 | 7 | 44% (7/16) |
| ELF ™ | Low | <7.7 | 0 | 0% (0/16) |

For NIS4™ and ELF™, there was a step wise increase in the proportion of «fast progressor» patients categorized from low to intermediate to high score zones. NIS4™ categorized more «fast progressor» patients into a high score zone (69%) as compared to ELF™ (56%). In contrast, FIB-4 and NFS classified the majority of the «fast progressor» patients into low or intermediate score zones.

The probability of progression to advanced fibrosis in 52 weeks by baseline score range was calculated (Table 3).

TABLE 3
Probability of fibrosis progression in 52 weeks by score.

| | | | | | Probability of |
|---|---|---|---|---|---|
| | | | | | Progression to |
| Score or | | | Patients | | F ≥ 3 in |
| Technology | Score Zone | Score Range | Per Zone | % of Total | 52 W |
| NIS4 ™ | High | ≥0.63 | 29 | 18% (29/161) | 38% (11/29) |
| NIS4 ™ | Indeterminate | 0.36 ≤ NIS4 < 0.63 | 45 | 28% (45/161) | 7% (3/45) |
| NIS4 ™ | Low | <0.36 | 87 | 54% (87/161) | 2% (2/87) |
| FIB-4 | High | ≥2.67 | 5 | 3% (5/161) | 80% (4/5) |
| FIB-4 | Indeterminate | 1.3 ≤ FIB-4 < 2.67 | 50 | 31% (50/161) | 18% (9/50) |
| FIB-4 | Low | <1.3 | 106 | 66% (106/161) | 3% (3/106) |
| NFS | High | ≥0.676 | 5 | 3% (5/161) | 40% (2/5) |
| NFS | Indeterminate | −1.455 ≤ NFS < 0.676 | 57 | 35% (57/161) | 14% (8/57) |
| NFS | Low | <−1.455 | 99 | 61% (99/161) | 6% (6/99) |
| ELF ™ | High | ≥9.8 | 28 | 17% (28/161) | 32% (9/28) |
| ELF ™ | Indeterminate | 7.7 ≤ ELF < 9.8 | 128 | 80% (128/161) | 5% (7/128) |
| ELF ™ | Low | <7.7 | 5 | 3% (5/161) | 0% (0/5) |

In this cohort, low score zones for tests evaluated generally highlighted lower risk of progression (0-6% in 52W) to advanced fibrosis (F≥3). On the other hand, high score zones generally conferred the highest risk of progression over the course of 52 weeks.

Finally, clinical performance to identify a «fast progressor» at the high cutoff for each test was calculated (Table 4). Diagnostic metrics (sensitivity, specificity, positive predictive value/PPV, negative predictive value/NPV) were provided with 95% Cl calculated with the asymptotic formula based on the normal approximation to the binomial distribution (Fleiss, 2003).

All statistical analyses were performed using R version 3.4.1 (R Core Team, 2017).

TABLE 4
Sensitivity and Specificity to identify a “fast progressor” calculated at the high cutoff for each
test evaluated.

| Score or | Score | Score | | | | |
|---|---|---|---|---|---|---|
| Technology | Zone | Range | Se | Sp | PPV | NPV |
| NIS4 ™ | High | ≥0.63 | 69% (44-86) | 88% (81-92) | 38% (23-56) | 96% (91-98) |
| FIB-4 | High | ≥2.67 | 25% (10-49) | 99% (86-100) | 80% (38-96) | 92% (87-96) |
| NFS | High | ≥0.676 | 13% (3-36) | 98% (94-99) | 40% (12-77) | 91% (86-95) |
| ELF ™ | High | ≥9.8 | 56% (33-77) | 87% (80-91) | 32% (18-51) | 95% (90-97) |

Se: sensitivity; Sp: specificity; PPV: positive predictive value; NPV: negative predictive value.

Overall, NIS4™ had the most balanced profile with the highest sensitivity (Se=69-83%) of the tests evaluated (Se=0%-67%) with an equally high specificity (Sp=88-89%).

NIS4 ≥0.63 correctly identified most (69%) of the «progressor» population as compared to FIB-4 ≥2.67 (25%), NFS ≥0.676 (13%), and ELF ≥9.8 (56%).

In conclusion, NIS4™ is able to identify the clinical subpopulation of «fast progressors» who have high likelihood of progression to advanced fibrosis within the course of 1 year. In addition, NIS4™ has a better prognostic utility than FIB-4, ELF and NAFLD fibrosis score to identify fast progressors patients.

1-6. (canceled)

7. A method for the identification of a subject as having Non-Alcoholic Fatty Liver Disease (NAFLD) with high likelihood of progression to advanced liver fibrosis within the course of 52 weeks, wherein said method comprises the measure of the level of hsa-miR34a, alpha 2 macroglobulin (A2M), YKL40 and glycated hemoglobin (HbA1c) in a blood-derived sample of said subject, the levels measured being used in a logistic function to calculate a score S, wherein the score S is calculated according to the following logistic function:
$$S ∼ \frac{{e}^{Y}}{1 + {e}^{Y}}$$
wherein:
Y=k+a*A+b*B+c*C+d*D
wherein the method is derived from the bootstrap model, and wherein:
S is the score;
A is the serum level of hsa-miR-34a in Cq;
B is the serum level of alpha 2 macroglobulin in g/L;
C is the serum level of YKL-40 in pg/ml;
D is the level of HbA1c in percent;
K is the constant of the logistic function;
a is a coefficient associated to the serum level of hsa-miR-34a;
b is a coefficient associated to the serum level of alpha 2 macroglobulin;
c is a coefficient associated to the serum level of YKL-40;
d is a coefficient associated to the level of HbA1c;
wherein:
k is a number comprised between 9.51 and 34.37;
a is a number comprised between −1.17 and −0.47;
b is a number comprised between 0.02 and 0.84;
c is a number comprised between 6.10E-06 and 2.09E-05; and
d is a number comprised between 0.07 and 0.89;
wherein the score S higher or equal to a cut off value, which is 0.63, is indicative of a high likelihood of progressing to advanced fibrosis within the course of 52 weeks.

8. The method according to claim 7, comprising the measure of the level of hsa-miR34a-5p, HbA1c, YKL-40 and A2M.

9. The method according to claim 7, wherein the subject has NAFLD with fibrosis stage 0, 1 or 2.

10. The method according to claim 7, further comprising the measure of liver fibrosis of said subject with a physical method.

11. The method according to claim 10, wherein measure of liver fibrosis with a physical method is carried out by measuring liver stiffness of said subject.

12. The method according to claim 11, wherein liver stiffness is measured by measuring the difference in velocity of elastic shear wave propagation in the liver.
